SEQUENCE LISTING (104 sequences)

SEQ ID NO: 1 - protein, 13 aa, Artificial Sequence
  CDR3s clonotypye1 TRa
CAADGGATNKLIF

SEQ ID NO: 2 - protein, 12 aa, Artificial Sequence
  CDR3s clonotype1 TRb
CASSQDRGEAFF

SEQ ID NO: 3 - protein, 17 aa, Artificial Sequence
  CDR3s clonotype2 TRa
CAVGTEAGGTSYGKLTF

SEQ ID NO: 4 - protein, 18 aa, Artificial Sequence
  CDR3s clonotype2 TRb
CASSPWGRLAGDLMTQYF

SEQ ID NO: 5 - protein, 12 aa, Artificial Sequence
  CDR3s clonotype3 TRb
CASSQDRGEAFF

SEQ ID NO: 6 - protein, 13 aa, Artificial Sequence
  CDR3s clonotype4 TRa
CAVQATGGFKTIF

SEQ ID NO: 7 - protein, 14 aa, Artificial Sequence
  CDR3s clonotype4 TRb
CSVDRGQVDYGYTF

SEQ ID NO: 8 - protein, 17 aa, Artificial Sequence
  CDR3s clonotype5 TRa
CAVGTEAGGTSYGKLTF

SEQ ID NO: 9 - protein, 18 aa, Artificial Sequence
  CDR3s clonotype6 TRb
CASSPWGRLAGDLMTQYF

SEQ ID NO: 10 - protein, 14 aa, Artificial Sequence
  CDR3s clonotype7 TRa
CAYKSGGGADGLTF

SEQ ID NO: 11 - protein, 13 aa, Artificial Sequence
  CDR3s clonotype7 TRb
CASSLPGAYEQYF

SEQ ID NO: 12 - protein, 11 aa, Artificial Sequence
  CDR3s clonotype8 TRa
CALTLNYQLIW

SEQ ID NO: 13 - protein, 15 aa, Artificial Sequence
  CDR3s clonotype8 TRb
CASSLGTSGYNEQFF

SEQ ID NO: 14 - protein, 14 aa, Artificial Sequence
  CDR3s clonotype9 TRa
CALSYSSNTGKLIF

SEQ ID NO: 15 - protein, 14 aa, Artificial Sequence
  CDR3s clonotype9 TRb
CASSLGQGSYEQYF

SEQ ID NO: 16 - protein, 12 aa, Artificial Sequence
  CDR3s clonotype10 TRa
CAVDNYGQNFVF

SEQ ID NO: 17 - protein, 17 aa, Artificial Sequence
  CDR3s clonotype10 TRb
CARSCRQGIIRNYGYTF

SEQ ID NO: 18 - protein, 17 aa, Artificial Sequence
  CDR3s clonotype10 TRb2
CASSLLPPTRLWDGYTF

SEQ ID NO: 19 - protein, 13 aa, Artificial Sequence
  VHHB11 11-4 CDR3s clonotype1 TRa
CAADGGATNKLIF

SEQ ID NO: 20 - protein, 12 aa, Artificial Sequence
  VHHB11 11-4 CDR3s clonotype1 TRb
CASSQDRGEAFF

SEQ ID NO: 21 - protein, 17 aa, Artificial Sequence
  VHHB11 11-4 CDR3s clonotype2 TRa
CAVGTEAGGTSYGKLTF

SEQ ID NO: 22 - protein, 18 aa, Artificial Sequence
  VHHB11 11-4 CDR3s clonotype2 TRb
CASSPWGRLAGDLMTQYF

SEQ ID NO: 23 - protein, 12 aa, Artificial Sequence
  VHHB11 11-4 CDR3s clonotype3 TRb
CASSQDRGEAFF

SEQ ID NO: 24 - protein, 17 aa, Artificial Sequence
  VHHB11 11-4 CDR3s clonotype4 TRa
CAVGTEAGGTSYGKLTF

SEQ ID NO: 25 - protein, 13 aa, Artificial Sequence
  VHHB11 11-4 CDR3s clonotype5 TRa
CAVQATGGFKTIF

SEQ ID NO: 26 - protein, 14 aa, Artificial Sequence
  VHHB11 11-4 CDR3s clonotype5 TRb
CSVDRGQVDYGYTF

SEQ ID NO: 27 - protein, 18 aa, Artificial Sequence
  VHHB11 11-4 CDR3s clonotype6 TRb
CASSPWGRLAGDLMTQYF

SEQ ID NO: 28 - protein, 14 aa, Artificial Sequence
  VHHB11 11-4 CDR3s clonotype7 TRa
CAYKSGGGADGLTF

SEQ ID NO: 29 - protein, 13 aa, Artificial Sequence
  VHHB11 11-4 CDR3s clonotype7 TRb
CASSLPGAYEQYF

SEQ ID NO: 30 - protein, 14 aa, Artificial Sequence
  VHHB11 11-4 CDR3s clonotype8 TRa
CALSYSSNTGKLIF

SEQ ID NO: 31 - protein, 14 aa, Artificial Sequence
  VHHB11 11-4 CDR3s clonotype8 TRb
CASSLGQGSYEQYF

SEQ ID NO: 32 - protein, 11 aa, Artificial Sequence
  VHHB11 11-4 CDR3s clonotype9 TRa
CALTLNYQLIW

SEQ ID NO: 33 - protein, 15 aa, Artificial Sequence
  VHHB11 11-4 CDR3s clonotype9 TRb
CASSLGTSGYNEQFF

SEQ ID NO: 34 - protein, 15 aa, Artificial Sequence
  VHHB11 11-4 CDR3s clonotype10 TRa
CAASMFAFGNEKLTF

SEQ ID NO: 35 - protein, 14 aa, Artificial Sequence
  VHHB11 11-4 CDR3s clonotype10 TRb
CASSPLGANTEAFF

SEQ ID NO: 36 - protein, 13 aa, Artificial Sequence
  VHAC1 1-8 CDR3s clonotype1 TRa
CILGMDSNYQLIW

SEQ ID NO: 37 - protein, 15 aa, Artificial Sequence
  VHAC1 1-8 CDR3s clonotype1 TRb
CASSQAHGQNQPQHF

SEQ ID NO: 38 - protein, 13 aa, Artificial Sequence
  VHAC1 1-8 CDR3s clonotype2 TRa
CILGMDSNYQLIW

SEQ ID NO: 39 - protein, 15 aa, Artificial Sequence
  VHAC1 1-8 CDR3s clonotype3 TRb
CASSQAHGQNQPQHF

SEQ ID NO: 40 - protein, 14 aa, Artificial Sequence
  VHAC1 1-8 CDR3s clonotype4 TRa
CAVRWETSGSRLTF

SEQ ID NO: 41 - protein, 15 aa, Artificial Sequence
  VHAC1 1-8 CDR3s clonotype4 TRb
CASSFGLAGPDTQYF

SEQ ID NO: 42 - protein, 15 aa, Artificial Sequence
  VHAC1 1-8 CDR3s clonotype5 TRb
CASSFGLAGPDTQYF

SEQ ID NO: 43 - protein, 14 aa, Artificial Sequence
  VHAC1 1-8 CDR3s clonotype6 TRa
CAVRWETSGSRLTF

SEQ ID NO: 44 - protein, 13 aa, Artificial Sequence
  VHAC1 1-8 CDR3s clonotype8 TRb
CASSPTANYGYTF

SEQ ID NO: 45 - protein, 14 aa, Artificial Sequence
  VHAC1 1-8 CDR3s clonotype7 TRa1
CAVRWETSGSRLTF

SEQ ID NO: 46 - protein, 13 aa, Artificial Sequence
  VHAC1 1-8 CDR3s clonotype7 TRa2
CILGMDSNYQLIW

SEQ ID NO: 47 - protein, 15 aa, Artificial Sequence
  VHAC1 1-8 CDR3s clonotype7 TRb
CASSFGLAGPDTQYF

SEQ ID NO: 48 - protein, 14 aa, Artificial Sequence
  VHAC1 1-8 CDR3s clonotype9 TRa1
CAVRWETSGSRLTF

SEQ ID NO: 49 - protein, 13 aa, Artificial Sequence
  VHAC1 1-8 CDR3s clonotype9 TRa2
CILGMDSNYQLIW

SEQ ID NO: 50 - protein, 15 aa, Artificial Sequence
  VHAC1 1-8 CDR3s clonotype9 TRb1
CASSFGLAGPDTQYF

SEQ ID NO: 51 - protein, 15 aa, Artificial Sequence
  VHAC1 1-8 CDR3s clonotype9 TRb2
CASSQAHGQNQPQHF

SEQ ID NO: 52 - protein, 14 aa, Artificial Sequence
  VHAC1 1-8 CDR3s clonotype10 TRa1
CAVRWETSGSRLTF

SEQ ID NO: 53 - protein, 13 aa, Artificial Sequence
  VHAC1 1-8 CDR3s clonotype10 TRa2
CILGMDSNYQLIW

SEQ ID NO: 54 - protein, 12 aa, Artificial Sequence
  VHAC1 1-9 CDR3s clonotype1 TRa
CAVMDSSYKLIF

SEQ ID NO: 55 - protein, 12 aa, Artificial Sequence
  VHAC1 1-9 CDR3s clonotype2 TRa
CAVLDSNYQLIW

SEQ ID NO: 56 - protein, 13 aa, Artificial Sequence
  VHAC1 1-9 CDR3s clonotype2 TRb
CASSDSDTDTQYF

SEQ ID NO: 57 - protein, 12 aa, Artificial Sequence
  VHAC1 1-9 CDR3s clonotype3 TRa
CAVLDSNYQLIW

SEQ ID NO: 58 - protein, 13 aa, Artificial Sequence
  VHAC1 1-9 CDR3s clonotype4 TRa
CILGMDSNYQLIW

SEQ ID NO: 59 - protein, 15 aa, Artificial Sequence
  VHAC1 1-9 CDR3s clonotype4 TRb
CASSQAHGQNQPQHF

SEQ ID NO: 60 - protein, 13 aa, Artificial Sequence
  VHAC1 1-9 CDR3s clonotype5 TRb
CASSDSDTDTQYF

SEQ ID NO: 61 - protein, 12 aa, Artificial Sequence
  VHAC1 1-9 CDR3s clonotype6 TRa
CAVMDSSYKLIF

SEQ ID NO: 62 - protein, 15 aa, Artificial Sequence
  VHAC1 1-9 CDR3s clonotype6 TRb
CASSEGGGGYEKLFF

SEQ ID NO: 63 - protein, 12 aa, Artificial Sequence
  VHAC1 1-9 CDR3s clonotype7 TRa
CGTAQGAQKLVF

SEQ ID NO: 64 - protein, 16 aa, Artificial Sequence
  VHAC1 1-9 CDR3s clonotype7 TRb
CASSFGDQRSGNTIYF

SEQ ID NO: 65 - protein, 12 aa, Artificial Sequence
  VHAC1 1-9 CDR3s clonotype8 TRa
CAVMDSSYKLIF

SEQ ID NO: 66 - protein, 13 aa, Artificial Sequence
  VHAC1 1-9 CDR3s clonotype8 TRb
CASSDSDTDTQYF

SEQ ID NO: 67 - protein, 15 aa, Artificial Sequence
  VHAC1 1-9 CDR3s clonotype9 TRb
CASSEGGGGYEKLFF

SEQ ID NO: 68 - protein, 15 aa, Artificial Sequence
  VHAC1 1-9 CDR3s clonotype10 TRb
CASSQAHGQNQPQHF

SEQ ID NO: 69 - protein, 13 aa, Artificial Sequence
  LP186-10-17 CDR3s clonotype1 TRa
CAVTGTQGGKLIF

SEQ ID NO: 70 - protein, 15 aa, Artificial Sequence
  LP186-10-17 CDR3s clonotype1 TRb
CASSLGTGVSTEAFF

SEQ ID NO: 71 - protein, 14 aa, Artificial Sequence
  LP186-10-17 CDR3s clonotype2 TRa
CILRDSNGANNLFF

SEQ ID NO: 72 - protein, 15 aa, Artificial Sequence
  LP186-10-17 CDR3s clonotype2 TRb
CASSPINRRNTEAFF

SEQ ID NO: 73 - protein, 13 aa, Artificial Sequence
  LP186-10-17 CDR3s clonotype3 TRa
CIPWHLNDYKLSF

SEQ ID NO: 74 - protein, 14 aa, Artificial Sequence
  LP186-10-17 CDR3s clonotype3 TRb
CASSFQGSGNTIYF

SEQ ID NO: 75 - protein, 14 aa, Artificial Sequence
  LP186-10-17 CDR3s clonotype4 TRa
CAASARTGANNLFF

SEQ ID NO: 76 - protein, 15 aa, Artificial Sequence
  LP186-10-17 CDR3s clonotype4 TRb
CASSDTSSYNSPLHF

SEQ ID NO: 77 - protein, 12 aa, Artificial Sequence
  LP186-10-17 CDR3s clonotype5 TRa1
CAVNKGYSTLTF

SEQ ID NO: 78 - protein, 16 aa, Artificial Sequence
  LP186-10-17 CDR3s clonotype5 TRa2
CVVRGLFSGGYNKLIF

SEQ ID NO: 79 - protein, 16 aa, Artificial Sequence
  LP186-10-17 CDR3s clonotype5 TRb
CASSSTGTGAAGELFF

SEQ ID NO: 80 - protein, 15 aa, Artificial Sequence
  LP186-10-17 CDR3s clonotype6 TRa
CAENSPNNAGNMLTF

SEQ ID NO: 81 - protein, 14 aa, Artificial Sequence
  LP186-10-17 CDR3s clonotype6 TRb
CASSQDAGNTEAFF

SEQ ID NO: 82 - protein, 16 aa, Artificial Sequence
  LP186-10-17 CDR3s clonotype7 TRa
CALSENSGGGADGLTF

SEQ ID NO: 83 - protein, 14 aa, Artificial Sequence
  LP186-10-17 CDR3s clonotype7 TRb
CASSFTEYQETQYF

SEQ ID NO: 84 - protein, 14 aa, Artificial Sequence
  LP186-10-17 CDR3s clonotype8 TRa
CVVNIGNYGQNFVF

SEQ ID NO: 85 - protein, 19 aa, Artificial Sequence
  LP186-10-17 CDR3s clonotype8 TRb
CASSASGTGGPRDTGELFF

SEQ ID NO: 86 - protein, 15 aa, Artificial Sequence
  LP186-10-17 CDR3s clonotype9 TRb
CASSYQTGASYGYTF

SEQ ID NO: 87 - protein, 13 aa, Artificial Sequence
  LP186-10-17 CDR3s clonotype10 TRa
CVVNTDSWGKLQF

SEQ ID NO: 88 - protein, 16 aa, Artificial Sequence
  LP186-10-17 CDR3s clonotype10 TRb
CASSWDRGAGANVLTF

SEQ ID NO: 89 - protein, 11 aa, Artificial Sequence
  LP186-02-18 CDR3s clonotype1 TRa
CARNTGNQFYF

SEQ ID NO: 90 - protein, 15 aa, Artificial Sequence
  LP186-02-18 CDR3s clonotype1 TRb
CASSYQTGASYGYTF

SEQ ID NO: 91 - protein, 11 aa, Artificial Sequence
  LP186-02-18 CDR3s clonotype2 TRa
CARNTGNQFYF

SEQ ID NO: 92 - protein, 16 aa, Artificial Sequence
  LP186-02-18 CDR3s clonotype2 TRb
CASSPLTGTGVYGYTF

SEQ ID NO: 93 - protein, 15 aa, Artificial Sequence
  LP186-02-18 CDR3s clonotype3 TRb
CASSYQTGASYGYTF

SEQ ID NO: 94 - protein, 16 aa, Artificial Sequence
  LP186-02-18 CDR3s clonotype4 TRb
CASSPLTGTGVYGYTF

SEQ ID NO: 95 - protein, 12 aa, Artificial Sequence
  LP186-02-18 CDR3s clonotype5 TRa
CVVNQAGTALIF

SEQ ID NO: 96 - protein, 13 aa, Artificial Sequence
  LP186-02-18 CDR3s clonotype5 TRb
CASSEQAFYEQYF

SEQ ID NO: 97 - protein, 15 aa, Artificial Sequence
  LP186-02-18 CDR3s clonotype6 TRa
CAENSPNNAGNMLTF

SEQ ID NO: 98 - protein, 14 aa, Artificial Sequence
  LP186-02-18 CDR3s clonotype6 TRb
CASSQDAGNTEAFF

SEQ ID NO: 99 - protein, 11 aa, Artificial Sequence
  LP186-02-18 CDR3s clonotype7 TRa
CARNTGNQFYF

SEQ ID NO: 100 - protein, 15 aa, Artificial Sequence
  LP186-02-18 CDR3s clonotype7 TRb
CASSYQTGAAYGYTF

SEQ ID NO: 101 - protein, 14 aa, Artificial Sequence
  LP186-02-18 CDR3s clonotype8 TRa
CILTPTHNTDKLIF

SEQ ID NO: 102 - protein, 12 aa, Artificial Sequence
  LP186-02-18 CDR3s clonotype8 TRb
CASSLLRQTQYF

SEQ ID NO: 103 - protein, 14 aa, Artificial Sequence
  LP186-02-18 CDR3s clonotype9 TRa
CAERLQTGANNLFF

SEQ ID NO: 104 - protein, 15 aa, Artificial Sequence
  LP186-02-18 CDR3s clonotype10 TRb
CASSYQTGAAYGYTF

(19) United States
(12) Patent Application Publication
(10) Pub. No.: US 2022/0170099 A1
(43) Pub. Date: Jun. 2, 2022

(54) IMMUNOME PROFILING FOR ENGINEERING WHITE BLOOD CELLS

(71) Applicant: Nantomics, LLC, Culver City, CA (US)

(72) Inventors: Shahrooz Rabizadeh, Agoura Hills, CA (US); Patrick Soon-Shiong, Culver City, CA (US); Peter Sieling, Culver City, CA (US); Stephen Charles Benz, Culver City, CA (US); Andrew Nguyen, Culver City, CA (US)

(21) Appl. No.: 17/629,574

(22) PCT Filed: Jul. 24, 2020
(86) PCT No.: PCT/US2020/043460

§ 371 Date: Jan. 24, 2022

Related U.S. Application Data
(60) Provisional application No. 62/878,656, filed on Jul. 25, 2019.

(51) Int. Cl. C12Q 1/6881; A61K 35/17; A61P 35/00; C07K 14/725; C12N 15/10; C12Q 1/6886

(52) U.S. Cl. CPC C12Q 1/6881; A61K 35/17; A61P 35/00; A61K 39/0011; C12N 15/1072; C12Q 1/6886; C07K 14/7051

(57) ABSTRACT

Single cell analysis from tumor tissue comprising tumor cells and immune competent cells and from peripheral white blood cells are used to obtain an immunome signature, and to gain information about the TCR repertoire. Such information is then employed to generate recombinant and patient specific therapeutic cells, including T cells (including T effector memory, T memory stem, naïve T, T central memory, CD8+ T, and CD4+ T cells), NK cells (cord-blood derived or PBMC derived or NK92), NKT cells, and dendritic cells.

This application claims priority to U.S. provisional application with the Ser. No. 62/878,656, filed Jul. 25, 2019, which is incorporated by reference herein.

### SEQUENCE LISTING

The content of the ASCII text file of the sequence listing named 102719.0015PCT_ST25, which is 26 KB in size was created on Jul. 17, 2019 and electronically submitted via EFS-Web along with the present application, and is incorporated by reference in its entirety.

### FIELD OF THE INVENTION

The field of the invention relates to systems and methods to identify patient specific treatment relevant molecules, especially as it relates to immunome related information and TCR diversity in the treatment of a tumor.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

While numerous systems and methods are known in the art to identify specific cells or molecules in a tumor, comprehensive analyses to detect immune status, immune cell types and activities as well as tumor specific TCR has been elusive. This is because such analysis is typically time and labor intensive beyond clinically relevant scales. Therefore, even though various systems and methods of tumor analyses are known in the art, such analyses tend to fall short of delivering a comprehensive data set that allows patient specific targeted treatments.

It is widely known that cancer cells in a host subject causes the subject to mount various humoral and cell-mediated immune responses comprised of T-cells and B-cells (including plasma cells) in an effort to remove the pathogen or tumor associated antigen (TAA). Following exposure, a portion of those T cells having a T cell receptor (TCR) targeting specific TAA are maintained in the host for many years without further antigenic exposure. This maintenance of specific T and B lymphocytes is referred to as immunological memory, the hallmark of which is the maintained ability of the host to mount rapid recall responses upon future tumor associated antigen encounter.

The establishment of immunological memory can take months to occur following initial antigenic encounter. Additionally, the mere establishment of immunological memory is not necessarily enough to confer protection against future encounters with a pathogen or foreign antigen, as a small memory population may be overwhelmed by a pathogen. Therefore, there is a need in the art to administer or establish a memory population large enough to provide the protection.

Cancer treatment, and especially personalized cancer treatment has increasingly become a viable option for many patients. However, despite such improved treatments, recurrence is still often not successfully managed and may lead to less than desirable outcomes. Among other reasons, tumor heterogeneity (see e.g., WO 2015/164560) significantly reduces chances of proper choice of antigens that will lead to treatment success. Moreover, as is described in WO 2014/058987 many tumors develop clonally different metastases over time and may therefore not be targeted by immune treatment. Still further, treatment with other non-immunotherapeutic drugs will interfere in most cases with immunotherapeutic drug treatment.

Therefore, even though various cancer treatment options for immunotherapy are known in the art, there still remains a need for systems and methods that help improve treatment outcome in immunotherapy of cancer, and specifically there is a need for new methods of cancer treatment that takes immunological memory into consideration.

### SUMMARY OF THE INVENTION

In one aspect, disclosed herein is a method of generating a treatment composition for a patient having a tumor, comprising: preparing from a tumor tissue a plurality of single cells comprising single tumor cells and single immune competent cells; using single cell nucleic analysis to determine from the plurality of single cells: (i) a T cell receptor profile for the immune competent cells; (ii) a first immune cell type profile; and using the T cell receptor profile and the immune cell type profile to generate recombinant white blood cells, wherein the recombinant white blood cells comprises T cell receptors targeting tumor associated antigens and neoepitopes, and wherein the neoepitopes are determined by tumor-normal sequencing. In some embodiments, the method further comprises a step of generating a second immune cell type profile using peripheral white blood cells.

In a second aspect, disclosed herein is a method of treating a patient having a tumor, comprising: obtaining a set of T cell receptor sequence information from a tumor tissue and a normal tissue of the patient, wherein each of the T cell receptor sequence information corresponds to one or more T cell receptors expressed in a single T cell; obtaining a set of single cell gene expression information from the tumor tissue and the normal tissue of the patient, wherein each of the single cell gene expression corresponds to gene expressions in a single while blood cell; determining, from the set of T cell receptor sequence information, a molecular profile of T cells in the tumor tissue by comparing the T cell receptor sequence information of the tumor tissue with the T cell receptor sequence information of the normal tissue; determining, from the set of single cell gene expression information, a molecular profile of white blood cells of the tumor tissue by comparing the single cell gene expression information of the tumor tissue with the and single cell gene expression information of the normal tissue; determining an immunome of the tumor tissue based on the molecular profiles of T cells and the white blood cells of the tumor tissue; and administering an immunotherapeutic composition comprising an immune competent cell that is genetically modified with a recombinant nucleic acid encoding a chimeric antigen receptor or a T cell receptor, wherein the recombinant nucleic acid comprises a nucleic acid segments encoding variable (V) and joining (J) segments selected based on the molecular profile of T cells.

In one embodiment of each of the above aspects, the T cell receptor sequence information is obtained from a single cell RNA-seq, and comprises a RNA sequence encoding variable (V), joining (J), and optionally diversity (D) segments of the T cell receptor. The V(D)J library preferably comprises a plurality of members, wherein each member comprises nucleic acid sequences encoding a barcode element, a unique molecular identifier (UMI), and a cDNA sequence reverse-transcribed from the RNA sequence.

In yet another aspect, disclosed herein is a method of profiling an immunome of a patient having a tumor, comprising: obtaining T cell receptor sequence information from a tumor tissue and a normal tissue of the patient, wherein each of the T cell receptor sequence information corresponds to one or more T cell receptors expressed in a single T cell; obtaining single cell gene expression information from the tumor tissue and the normal tissue of the patient, wherein each of the single cell gene expression corresponds to gene expressions in a single white blood cell; determining, from the T cell receptor sequence information, a molecular profile of T cells in the tumor tissue by comparing the T cell receptor sequence information of the tumor tissue with the T cell receptor sequence information of the normal tissue; determining, from the set of single cell gene expression information, a molecular profile of white blood cells of the tumor tissue by comparing the single cell gene expression information of the tumor tissue with the and single cell gene expression information of the normal tissue; and determining an immunome of the tumor tissue based on the molecular profiles of T cells and the white blood cells of the tumor tissue.

In one embodiment of each of the above aspects, the molecular profile of T cells comprises at least one of number of cells expressing T cell receptor, a number of clonotype, and a frequency of the clonotype. The single cell gene expression information is obtained from single cell RNA-seq of a plurality of genes, each the gene encoding a protein in an immune response pathway. The method may further comprise constructing a gene expression library having a plurality of members, wherein each member comprises nucleic acid sequences encoding a barcode element, a unique molecular identifier (UMI), and a cDNA sequence reverse-transcribed from the RNA sequence of the plurality of genes. The the molecular profile of white blood cells comprises a median number of genes expressed per cell, total number of detected genes, and median number of the unique molecular identifier. The method may further comprise clustering white blood cells in the tumor tissue into a plurality of clusters based on the molecular profile.

In one embodiment, the method further comprises determining expressions of an immune cell marker gene. The immune cell marker gene may comprise CD3G, CD4, CD8A, NCAM1 (CD56), FCGR3A (CD16), NCR1 (NK-p46), IFN-γ, TGF-β1, FOXP3, LAG3, and SNAP47.

In one preferred embodiment, the method may further comprise creating an immunotherapeutic composition comprising an immune competent cell that is genetically modified with a recombinant nucleic acid encoding a chimeric antigen receptor or a T cell receptor; and wherein the recombinant nucleic acid comprises a nucleic acid segments encoding variable (V) and joining (J) segments selected based on the molecular profile of T cells. The immune competent cell is contemplated to be a T cell, an NK cell, a genetically engineered NK cell, or an NKT cell. The method may further include administering a plurality of immune competent cells to the patient, wherein types of the plurality of immune competent cells are selected based on the molecular profile of the white blood cells. Preferably, at least one of the immune competent cells is the patient's autologous cell.

### BRIEF DESCRIPTION OF THE DRAWING

FIG. 1 is an exemplary illustration of cDNA amplification of samples.

FIG. 2 is an exemplary illustration of V(D)J sequencing libraries of samples

FIG. 3 is an exemplary illustration of V(D)J library structure.

FIG. 4 is an exemplary illustration of GEX sequencing libraries of samples

FIG. 5 is an exemplary illustration of gene expression library structure.

FIG. 6 is an exemplary illustration of single transcript analysis. (A) 6204 cells 9 different clusters; (B) 6204 cells CD3G; (C) CD4; (D) CD8A; (E) NCAM1 CD56; (F) FCGR3A CD16; (G) NCR1 NK-p46; (H) IFNγ; (I) TGFβ1; (J) FOXP3; (K) LAG3; and (L) SNAP47.

### DETAILED DESCRIPTION

The inventors have now discovered that single cell analysis of tumor and normal tissues can be employed to obtain comprehensive data set that allows development of patient specific targeted treatments. In preferred aspects, contemplated methods use the isolation of single cells from a tumor sample for individualized molecular characterization (e.g., by sequencing) to better understand and derive individualized treatments for a patient. More specifically, preferred analyses is related to the molecular characterization of a cancer patient's immunome as discovered through analysis of their tumor sample relative to a normal sample from the patient, as well as by characterization of the patient's white blood cells independent of a tumor sample.

The characterization of the tumor/normal samples in the workflow is used to derive individualized novel treatments for the patient, in a manner wherein the molecular information serves as the blueprint used to engineer the patient's own white blood cells such as T cells (including T effector memory, T memory stem, naïve T, T central memory, CD8+ T, and CD4+ T cells), NK cells (cord-blood derived or PBMC derived), NKT cells, and dendritic cells or allogeneic off-the-shelf cells (e.g., NK-92), and are then used to treat the individual patient. Expression analyses (RNA or protein) of barcoded single cells in a large batch of tumor and immune cells are used to derive such important information as the identification of the T cell receptors expressed in the tumor and the tumor microenvironment as well as in circulating blood, and the prevalence of different types of immunological cells in the tumor and/or tumor microenvironment and in circulation.

In one embodiment, the present disclosure contemplates the isolation of single cells from a tumor sample for individualized molecular characterization. Such molecular characterization may be done by sequencing, including whole genome sequencing and RNA sequencing. The molecular characterization of the single cells from the tumor sample, thus obtained, is used to better understand and derive novel, individualized treatments for a patient. Put another way, the application herein is related to the molecular characterization of a cancer patient's immunome as discovered through analysis of their tumor sample relative to a normal sample from the patient, as well as by characterization of the patient's white blood cells independent of a tumor sample.

In various embodiments of this present disclosure, the inventors provide techniques for using single cell analysis as a path to immunological memory and cancer cure. The cell source may be tumor tissue, blood, Cerebrospinal fluid (CSF), and Peritoneal cavity fluid (ascites). The cells may be taken from the individual at diagnosis of a tumor, following tumor treatment, or for continuous monitoring during and after treatment. The cell source may also be a normal tissue

Single cell genomics are contemplated herein because such a method enables the understanding of cell to cell differences and cellular heterogenicity, which is masked in bulk sequencing and RNA-seq methods. Methods of doing single cell RNA seq are commercially available, for example from 10× genomics, and such techniques are contemplated to be used in the instantly disclosed methods. Briefly, single cells, reverse transcription (RT) reagents, Gel Beads containing barcoded oligonucleotides, and oil are combined on a microfluidic chip to form reaction vesicles called Gel Beads in Emulsion, or GEMs. GEMs may be formed in parallel within the microfluidic channels of the chip, allowing the user to process 100's to 10,000's of single cells concurrently.

Each functional GEM is contemplated to contain a single cell, a single Gel Bead, and RT reagents. Within each GEM reaction vesicle, a single cell is lysed, the Gel Bead is dissolved to free the identically barcoded RT oligonucleotides into solution, and reverse transcription of polyadenylated mRNA occurs. As a result, all cDNAs from a single cell will have the same barcode, allowing the sequencing reads to be mapped back to their original single cells of origin. The preparation of NGS libraries from these barcoded cDNAs is then carried out in a highly efficient bulk reaction.

By using the above described single cell genomics, single cell TCR profiling of cancer versus normal cells and single cell immune subclass profiling (white cell profiling) were done to determine TCR targeting TAAs and neoepitopes.

In this context, it should be appreciated that preferred neoepitopes are not epitopes that are common to cancers (e.g., CEA) or epitopes that are specific to a particular type of cancer (e.g., PSA), but antigens that are exclusive to the particular tumor or even location within the tumor. Moreover, the neoepitopes contemplated herein are also specific to the particular patient (thus eliminating SNPs and other known variants), and also specific with respect to their anatomical location. Viewed from a different perspective, contemplated neoepitopes are genuine to the specific patient and his/her HLA-type, the tumor, and the location. In addition, neoepitopes may further be specific to a particular treatment phase (e.g., prior to treatment, subsequent to a first round of treatment, etc.).

False positives in the neoepitope population, i.e., neoepitopes having no therapeutic effect, may be eliminated by using the methods described in U.S. Pat. No. 10,532,089, which is incorporated by reference herein in its entirety. In brief, neoepitopes are selected by the steps of (a) receiving omics data for tumor cells in a first location in a patient, and receiving omics data for tumor cells in a second location in a patient; (b) using the omics data to determine respective neoepitopes in the tumor cells of the first and second locations; (c) identifying treatment relevant neoepitopes in the tumor cells of the first and second locations using at least one of a group attribute, a location attribute, and a function attribute.

Neoepitopes may be identified by considering the type (e.g., deletion, insertion, transversion, transition, translocation) and impact of the mutation (e.g., non-sense, missense, frame shift, etc.), which may as such serve as a first content filter through which silent and other non-relevant (e.g., non-expressed) mutations are eliminated. The neoepitope sequences can be defined as sequence stretches with relatively short length (e.g., 7-11 mers) wherein such stretches will include the change(s) in the amino acid sequences. Most typically, the changed amino acid will be at or near the central amino acid position. For example, a typical neoepitope may have the structure of A₄-N-A₄, or A₃-N-A₅, or A₂-N-A₇, or A₅-N-A₃, or A₇-N-A₂, where A is a proteinogenic amino acid and N is a changed amino acid (relative to wild type or relative to matched normal). For example, neoepitope sequences as contemplated herein include sequence stretches with relatively short length (e.g., 5-30 mers, more typically 7-11 mers, or 12-25 mers) wherein such stretches include the change(s) in the amino acid sequences.

Thus, a single amino acid change may be presented in numerous neoepitope sequences that include the changed amino acid, depending on the position of the changed amino acid. Advantageously, such sequence variability allows for multiple choices of neoepitopes and so increases the number of potentially useful targets that can then be selected on the basis of one or more desirable traits (e.g., highest affinity to a patient HLA-type, highest structural stability, etc.). Most typically, such neoepitopes will be calculated to have a length of between 2-50 amino acids, more typically between 5-30 amino acids, and most typically between 9-15 amino acids, with a changed amino acid preferably centrally located or otherwise situated in a manner that allows for or improves its binding to MHC. For example, where the epitope is to be presented by the MHC-I complex, a typical neoepitope length will be about 8-11 amino acids, while the typical neoepitope length for presentation via MHC-II complex will have a length of about 13-17 amino acids. Since the position of the changed amino acid in the neoepitope may be other than central, the actual peptide sequence and with that actual topology of the neoepitope may vary considerably.

In various embodiment, the discovery of neoepitopes may start with a variety of biological materials, including fresh biopsies, frozen or otherwise preserved tissue or cell samples, circulating tumor cells, exosomes, various body fluids (and especially blood), etc. as is further discussed in more detail below. Thus, suitable methods of omics analysis include nucleic acid sequencing, and particularly single cell GEMS, NGS methods operating on DNA (e.g., Illumina sequencing, ion torrent sequencing, 454 pyrosequencing, nanopore sequencing, etc.), RNA sequencing (e.g., RNAseq, reverse transcription based sequencing, etc.), and protein sequencing or mass spectroscopy based sequencing (e.g., SRM, MRM, CRM, etc.).

As such, and particularly for nucleic acid based sequencing, it should be particularly recognized that high-throughput genome sequencing of a tumor tissue will allow for rapid identification of neoepitopes. However, it must be appreciated that where the so obtained sequence information is compared against a standard reference, the normally occurring inter-patient variation (e.g., due to SNPs, short indels, different number of repeats, etc.) as well as heterozygosity will result in a relatively large number of potential false positive neoepitopes. Notably, such inaccuracies can be eliminated where a tumor sample of a patient is compared against a matched normal (i.e., non-tumor) sample of the same patient.

In one especially preferred aspect of the inventive subject matter, DNA and RNA analysis is performed by whole genome sequencing, whole transcriptome sequencing, and/or exome sequencing (typically at a coverage depth of at least 10×, more typically at least 20×) of both tumor and matched normal sample. Alternatively, DNA data may also be provided from an already established sequence record (e.g., SAM, BAM, FASTA, FASTQ, or VCF file) from a prior sequence determination. Therefore, data sets may include unprocessed or processed data sets, and exemplary data sets include those having BAMBAM format, SAMBAM format, FASTQ format, or FASTA format. However, it is especially preferred that the data sets are provided in BAMBAM format or as BAMBAM diff objects (see e.g., US2012/0059670A1 and US2012/0066001A1). Moreover, it should be noted that the data sets are reflective of a tumor and a matched normal sample of the same patient to so obtain patient and tumor specific information. Thus, genetic germ line alterations not giving rise to the tumor (e.g., silent mutation, SNP, etc.) can be excluded. Of course, and addressed in more detail below, it should be recognized that the tumor sample may be from an initial tumor, from the tumor upon start of treatment, from a recurrent tumor or metastatic site, etc. In most cases, the matched normal sample of the patient may be blood, or non-diseased tissue from the same tissue type as the tumor.

Of course, it should be noted that the computational analysis of the sequence data may be performed in numerous manners. In most preferred methods, however, analysis is performed in silico by location-guided synchronous alignment of tumor and normal samples as, for example, disclosed in US 2012/0059670A1 and US 2012/0066001A1 using BAM files and BAM servers. Such analysis advantageously reduces false positive neoepitopes and significantly reduces demands on memory and computational resources.

Once patient/tumor specific neoepitopes are identified, computational analysis can be performed by docking neoepitopes to the HLA and determining best binders (e.g., lowest K_{D}, for example, less than 500 nM, or less than 250 nM, or less than 150 nM, or less than 50 nM), for example, using NetMHC. It should be appreciated that such approach will not only identify specific neoepitopes that are genuine to the patient and tumor for each location, but also those neoepitopes that are most likely to be presented on a cell and as such most likely to elicit an immune response with therapeutic effect. Of course, it should also be appreciated that thusly identified HLA-matched neoepitopes can be biochemically validated in vitro (e.g., to establish high-affinity binding between MHC complex and neoepitope and/or presentation) prior to use in a therapeutic composition.

In further contemplated aspects, verification of potential neoepitope presentation may also be performed using neoepitopes that are preferably labeled with an affinity marker or entity for optical detection. Such neoepitopes may be useful in detecting binding of the neoepitope to T-cell receptors, MHC complexes, etc. In addition, and particularly where such neoepitopes are coupled to a solid phase, the neoepitopes may be used to detect and isolate antibodies from the patient that may already be present.

The single cell analysis, and in particular the GEM analysis disclosed above, is also used to identify T memory stem cells. Memory T cells are long-lived T cells, that remains in the body for rapid response upon pathogen re-exposure. Because memory T cells have been trained to recognize specific antigens, they will trigger a faster and stronger immune response after encountering the same antigen. Maximizing T cell memory, as disclosed in US Patent Application Publication No. 2020/0023008 is also contemplated herein.

Once neoepitopes and T-cell receptors targeting tumor associated antigens are discovered by tumor and normal cell sequencing and by RNASeq, autologous white blood cells are engineered with such neoepitopes and T-cell receptors targeting tumor associated antigens. The autologous white blood cells may comprise naïve T cells, T memory stem cells, T central memory cells, T effector memory cells, CD8+ T cells, CD4+ T cells, NK cells, NKT cells, Dendritic Cells, NK-92 (allogeneic), cord blood derived cells. Electroporation methods are generally used to engineer the autologous white blood cells with nucleotide vectors comprising the one or more neoepitopes and T cell receptors.

In a preferred embodiment, the electroporation systems and methods of transfection of mammalian cells used herein are disclosed in US Patent Application Publication No.: US20180100161A1, which is incorporated by reference in its entirety. In this electroporation protocol, the cells are subjected to multiple pulses at a moderate voltage, a small gap width, relatively moderate capacitance, and a short time constant.

Immune competent cells can be transfected with RNA (e.g., synthetic RNA, mRNA, in vitro transcribed RNA, etc.) using multi-pulse conditions using a very short time constant, typically a time constant of less than 10 msec, or even more typically of less than 5 msec. For example, the time constant may range from about 0.5 to 10 ms, from about 1 to 5 ms, and from about 1 to 4 ms; most typically the time constant is between 1-3 msec. Such conditions are generally achieved using a cell gap of 0.2 cm and a voltage of about 200V. Viewed from another perspective, the field strength of electroporation is typically between about 800 V/cm and 1200 V/cm. However, lower field strengths (e.g., about 600-800 V/cm, or about 400-600 V/cm) and higher field strengths (e.g., about 1,000-1,400 V/cm) are also contemplated. Therefore, the gap width need not be limited to 0.2 cm, but may also range from about 0.1 cm to 0.4 cm. The amount of mRNA added to the electroporation reaction may be about 600 ng, about 1000 ng, or more.

With respect to suitable capacitance, it is contemplated that the capacitance should be relatively moderate, typically about 10 μF, and more typically about 25 μF. Viewed form a different perspective, suitable capacitance settings will be between about 1 to 150 μF or about 1-100 μF, and more typically between about 5-75 μF, or about 5-50 μF, about 10 to 40 μF, or about 20-30 μF. Both high voltage with low capacitance (short pulse duration) or low voltage with high capacitance (long pulse duration) have previously been used to achieve successful gene transfer (Nucl Acids Res. 1987; 15:1311-1326). Notably, the present systems and methods use a low voltage moderate capacitance setting to achieve high transfection efficiency at high viability in a relatively conductive electroporation medium.

With respect to suitable pulse numbers and pulse-to-pulse intervals, the inventors noted that at least two, three, and in some cases four pulses provided more desirable results than a single pulse or of five or more pulses. Therefore, it is contemplated that a preferred pulse number is between 2-4 pulses. Most typically, the pulses are separated from subsequent pulses by a relatively short interval, typically between 1 second and 15 seconds, and in some cases even longer. However, interval lengths of between 2-10 seconds are generally preferred.

The medium in which the cells are transfected is an isotonic medium, optionally containing one or more nutrients. Therefore, and viewed from a different perspective, suitable media include growth media (with or without serum), and especially RPMI, MEM, and DMEM. In some aspects, the medium is RPMI, a high-conductivity medium, wherein the conductivity of RPMI is about 1370 mS/m. Media also may include minimal media and Ringer's solution. Thus, it should be noted that the media are generally electrically conductive media. In other aspects, the medium may also be sterile (and in some cases non-isotonic) non- or low-conductance. solutions.

The thusly engineered cells are expanded by various methods such as GPM-in-a-Box, in cytokine mixture of IL7, IL15, and IL21 to establish T memory stem cells. The expanded and engineered T cells are then administered to the patient. In a preferred embodiment, a Nant cancer vaccine as disclosed in WO2018005973A1, which is incorporated by reference, may be administered in combination with the engineered cells disclosed herein.

Methods of administration include, but are not limited to, intravenous, intratumoral, intradermal, intramuscular, intraperitoneal, subcutaneous, epidural, sublingual, intracerebral, intraventricular, intrathecal, etc. Additional examples of suitable modes of administration are well known in the art. Compositions for parenteral administration may be enclosed in ampoule, a disposable syringe or a multiple-dose vial made of glass, plastic or other material.

Embodiments of the present disclosure are further described in the following examples. The examples are merely illustrative and do not in any way limit the scope of the invention as claimed.

### EXAMPLES

### Example 1: V(D)J Sequencing

The inventors investigated eight different samples for this study, as illustrated in Table 1 below. Biopsy specimens were minced to single cell suspensions. The suspensions were cultured in human serum and T-cell growth factors (IL-2, IL-7 an IL-15). Multiple cultures were initiated with multiple pieces of tissue.

TABLE 1

| Name of the sample | Cancer type | Cells Assayed |
|---|---|---|
| VHHB11 11-3 | Gall Bladder | TILs and other cells |
| | | from the tumor tissue |
| VHHB11 11-4 | Gall Bladder | As above |
| VHAC1-1-1 | Colon | As above |
| VHAC1 1-8 | Colon | As above |
| VHAC1 1-9 | Colon | As above |
| LP186 10-17 | Healthy subject draw 1 | PBMCs |
| LP186 02-18 | Healthy subject draw 2 | PBMCs |
| LP381 02-18 | Healthy subject | PBMCs |

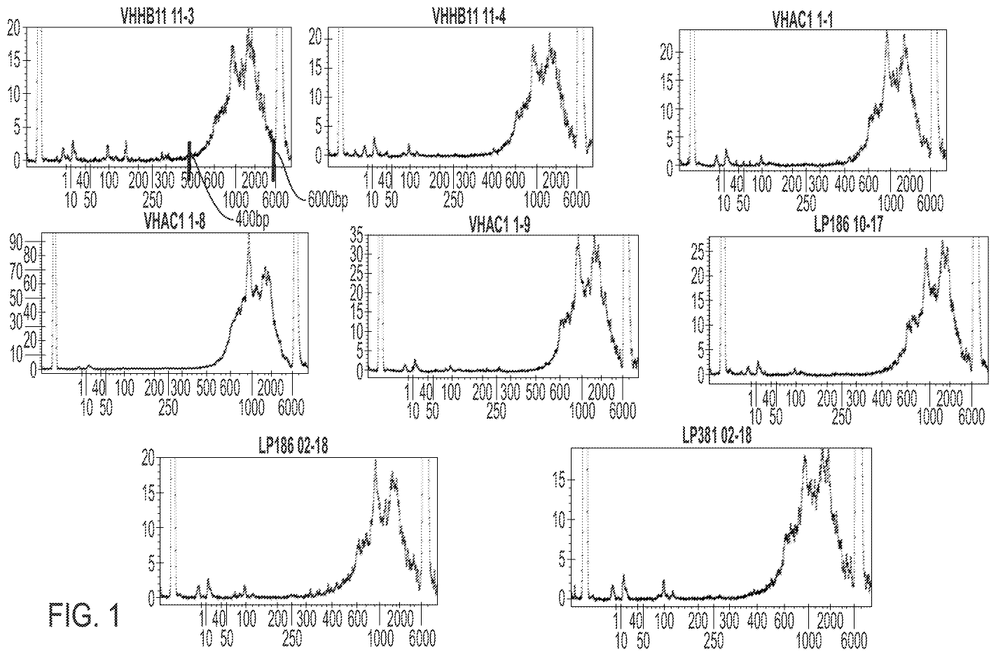

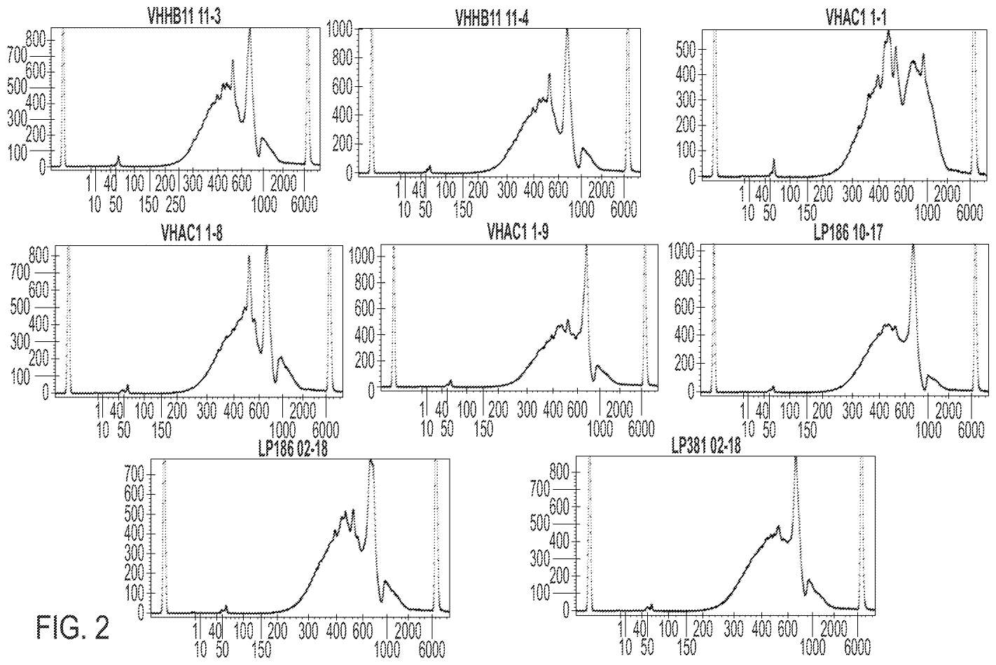

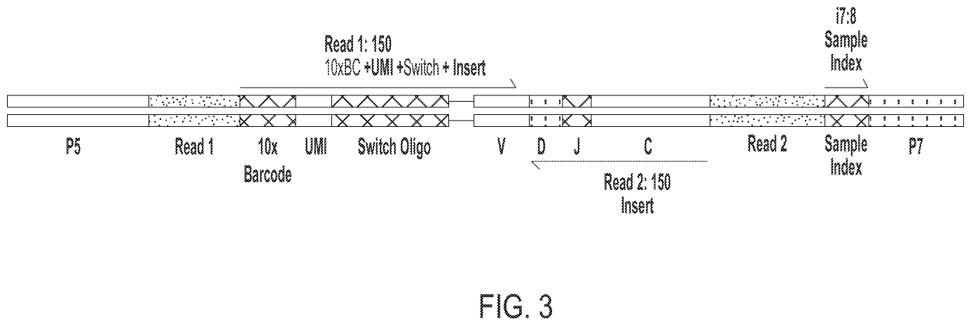

FIG. 1 illustrates cDNA amplifications from aforementioned 8 samples, and the V(D)J sequencing libraries for these 8 samples are shown in FIG. 2 In that regard, it should be noted that V(D)J recombination is the process by which T cells and B cells randomly assemble different gene segments—known as variable (V), diversity (D) and joining (J) genes—in order to generate unique receptors (known as antigen receptors) that can collectively recognize many different types of molecule. The V(D)J library structure is shown in FIG. 3.

Table 2 shows a summary of V(D)J sequencing results for the right samples. Top 10 clonotypes for samples VHHB11 11-3, VHHB11 11-4, VHAC1 1-8, VHAC1 1-9, LP186 10-17 and LP381 02-18 are illustrated in Tables 3-8 respectively.

TABLE 2

| | | | | Clonotype | Clonotype |
|---|---|---|---|---|---|
| | | No. | | with a | with a |
| | Estimated | of cells | Total | frequency | frequency |
| Name of the | no. of | with V-J | number of | of at | of at |
| sample | cells | spanning | clonotypes | least 6 | least 10 |
| VHHB11 | 6808 | 3898 (57%) | 829 | 72 | 50 |
| 11-3 | | | | | |
| VHHB11 | 7730 | 4700 (61%) | 893 | 85 | 49 |
| 11-4 | | | | | |
| VHAC1-1-1 | 5752 | 41 | Failed | Failed | Failed |
| | | | prep | prep | prep |
| VHAC1 1-8 | 6809 | 3218 (47%) | 50 | 6 | 6 |
| VHAC1 1-9 | 12496 | 4545 (36%) | 234 | 30 | 22 |
| LP186 10-17 | 5729 | 4119 (72%) | 1506 | 138 | 88 |
| LP186 02-18 | 5294 | 3452 (65%) | 855 | 69 | 44 |
| LP381 02-18 | No data | No data | No data | No data | No data |

TABLE 3
TOP 10 CLONOTYPES IN SAMPLE - VHHB11 11-3

| Clonotype | | | |
|---|---|---|---|
| ID | CDR3s | Proportion | Frequency |
| clonotype1 | TRα: CAADGGATNKLIF | 18.80% | 1,281 |
| | (SEQ ID NO: l) | | |
| | TRβ: CASSQDRGEAFF | | |
| | (SEQ ID NO: 2) | | |
| clonotype2 | TRα: CAVGTEAGGTSYGK | 10.70% | 729 |
| | LTF (SEQ ID NO: 3) | | |
| | TRβ: CASSPWGRLAGDLM | | |
| | TQYF (SEQ ID NO: 4) | | |
| clonotype3 | TRβ: CASSQDRGEAFF | 8.40% | 572 |
| | (SEQ ID NO: 5) | | |
| clonotype4 | TRα: CAVQATGGFKTIF | 3.20% | 217 |
| | (SEQ ID NO: 6) | | |
| | TRβ: CSVDRGQVDYGYTF | | |
| | (SEQ ID NO: 7) | | |
| clonotype5 | TRα: CAVGTEAGGTSYGK | 2.90% | 197 |
| | LTF (SEQ ID NO: 8) | | |
| clonotype6 | TRβ: CASSPWGRLAGDLM | 2.10% | 141 |
| | TQYF (SEQ ID NO: 9) | | |
| clonotype7 | TRα: CAYKSGGGADGLTF | 1.30% | 86 |
| | (SEQ ID NO: 10) | | |
| | TRβ: CASSLPGAYEQYF | | |
| | (SEQ ID NO: 11) | | |
| clonotype8 | TRα: CALTLNYQLIW | 1.10% | 77 |
| | (SEQ ID NO: 12) | | |
| | TRβ: CASSLGTSGYNEQF | | |
| | F (SEQ ID NO: 13) | | |
| clonotype9 | TRα: CALSYSSNTGKLIF | 1.10% | 77 |
| | (SEQ ID NO: 14) | | |
| | TRβ: CASSLGQGSYEQYF | | |
| | (SEQ ID NO: 15) | | |
| clonotype10 | TRα: CAVDNYGQNFVF | 1.00% | 71 |
| | (SEQ ID NO: 16) | | |
| | TRβ: CARSCRQGIIRNYG | | |
| | YTF (SEQ ID NO: 17) | | |
| | TRβ: CASSLLPPTRLWDG | | |
| | YTF (SEQ ID NO: 18) | | |

TABLE 4
TOP 10 CLONOTYPES IN SAMPLE - VHHB11 11-4

| Clonotype | | | |
|---|---|---|---|
| ID | CDR3s | Proportion | Frequency |
| clonotype1 | TRα: CAADGGATNKLIF | 18.20% | 1,408 |
| | (SEQ ID NO: 19) | | |
| | TRβ: CASSQDRGEAFF | | |
| | (SEQ ID NO: 20) | | |
| clonotype2 | TRα: CAVGTEAGGTSYGK | 13.30% | 1,025 |
| | LTF (SEQ ID NO: 21) | | |
| | TRβ: CASSPWGRLAGDLM | | |
| | TQYF (SEQ ID NO: | | |
| | 22) | | |
| clonotype3 | TRβ: CASSQDRGEAFF | 7.30% | 562 |
| | (SEQ ID NO: 23) | | |
| clonotype4 | TRα: CAVGTEAGGTSYGK | 3.00% | 233 |
| | LTF (SEQ ID NO: 24) | | |
| clonotype5 | TRα: CAVQATGGFKTIF | 3.00% | 231 |
| | (SEQ ID NO: 25) | | |
| | TRβ: CSVDRGQVDYGYTF | | |
| | (SEQ ID NO: 26) | | |
| clonotype6 | TRβ: CASSPWGRLAGDLM | 2.10% | 161 |
| | TQYF (SEQ ID NO: | | |
| | 27) | | |
| clonotype7 | TRα: CAYKSGGGADGLTF | 1.50% | 119 |
| | (SEQ ID NO: 28) | | |
| | TRβ: CASSLPGAYEQYF | | |
| | (SEQ ID NO: 29) | | |
| clonotype8 | TRα: CALSYSSNTGKLIF | 1.40% | 104 |
| | (SEQ ID NO: 30) | | |
| | TRβ: CASSLGQGSYEQYF | | |
| | (SEQ ID NO: 31) | | |
| clonotype9 | TRα: CALTLNYQLIW | 1.10% | 89 |
| | (SEQ ID NO: 32) | | |
| | TRβ: CASSLGTSGYNEQF | | |
| | F (SEQ ID NO: 33) | | |
| clonotype10 | TRα: CAASMFAFGNEKLT | 1.10% | 86 |
| | F (SEQ ID NO: 34) | | |
| | TRβ: CASSPLGANTEAFF | | |
| | (SEQ ID NO: 35) | | |

TABLE 5
TOP 10 CLONOTYPES IN SAMPLE - VHAC1 1-8

| Clonotype | | | |
|---|---|---|---|
| ID | CDR3s | Proportion | Frequency |
| clonotype1 | TRα: CILGMDSNYQLIW | 46.60% | 3,171 |
| | (SEQ ID NO: 36) | | |
| | TRβ: CASSQAHGQNQPQH | | |
| | F (SEQ ID NO: 37) | | |
| clonotype2 | TRα: CILGMDSNYQLIW | 41.00% | 2,790 |
| | (SEQ ID NO: 38) | | |
| clonotype3 | TRβ: CASSQAHGQNQPQH | 3.30% | 221 |
| | F (SEQ ID NO: 39) | | |
| clonotype4 | TRα: CAVRWETSGSRLTF | 0.40% | 29 |
| | (SEQ ID NO: 40) | | |
| | TRβ: CASSFGLAGPDTQY | | |
| | F (SEQ ID NO: 41) | | |
| clonotype5 | TRβ: CASSFGLAGPDTQY | 0.30% | 17 |
| | F (SEQ ID NO: 42) | | |
| clonotype6 | TRα: CAVRWETSGSRLTF | 0.20% | 12 |
| | (SEQ ID NO: 43) | | |
| clonotype8 | TRβ: CASSPTANYGYTF | 0.00% | 3 |
| | (SEQ ID NO: 44) | | |
| clonotype7 | TRα: CAVRWETSGSRLTF | 0.00% | 3 |
| | (SEQ ID NO: 45) | | |
| | TRα: CILGMDSNYQLIW | | |
| | (SEQ ID NO: 46) | | |
| | TRβ: CASSFGLAGPDTQY | | |
| | F (SEQ ID NO: 47) | | |
| clonotype9 | TRα: CAVRWETSGSRLTF | 0.00% | 2 |
| | (SEQ ID NO: 48) | | |
| | TRα: CILGMDSNYQLIW | | |
| | (SEQ ID NO: 49) | | |
| | TRβ: CASSFGLAGPDTQY | | |
| | F (SEQ ID NO: 50) | | |
| | TRβ: CASSQAHGQNQPQH | | |
| | F (SEQ ID NO: 51) | | |
| clonotype10 | TRα: CAVRWETSGSRLTF | 0.00% | 2 |
| | (SEQ ID NO: 52) | | |
| | TRα: CILGMDSNYQLIW | | |
| | (SEQ ID NO: 53) | | |

TABLE 6
TOP 10 CLONOTYPES IN SAMPLE - VHAC1 1-9

| Clonotype | | | |
|---|---|---|---|
| IDs | CDR3s | Proportion | Frequency |
| clonotype1 | TRα: CAVMDSSYKLIF | 22.40% | 2,796 |
| | (SEQ ID NO: 54) | | |
| clonotype2 | TRα: CAVLDSNYQLIW | 17.50% | 2,190 |
| | (SEQ ID NO: 55) | | |
| | TRβ: CASSDSDTDTQYF | | |
| | (SEQ ID NO: 56) | | |
| clonotype3 | TRα: CAVLDSNYQLIW | 12.90% | 1,611 |
| | (SEQ ID NO: 57) | | |
| clonotype4 | TRα: CILGMDSNYQLIW | 6.10% | 766 |
| | (SEQ ID NO: 58) | | |
| | TRβ: CASSQAHGQNQPQH | | |
| | F (SEQ ID NO: 59) | | |
| clonotype5 | TRβ: CASSDSDTDTQYF | 6.00% | 748 |
| | (SEQ ID NO: 60) | | |
| clonotype6 | TRα: CAVMDSSYKLIF | 5.90% | 732 |
| | (SEQ ID NO: 61) | | |
| | TRβ: CASSEGGGGYEKLF | | |
| | F (SEQ ID NO: 62) | | |
| clonotype7 | TRα: CGTAQGAQKLVF | 2.40% | 303 |
| | (SEQ ID NO: 63) | | |
| | TRβ: CASSFGDQRSGNTI | | |
| | YF (SEQ ID NO: 64) | | |
| clonotype8 | TRα: CAVMDSSYKLIF | 1.40% | 174 |
| | (SEQ ID NO: 65) | | |
| | TRβ: CASSDSDTDTQYF | | |
| | (SEQ ID NO: 66) | | |
| clonotype9 | TRβ: CASSEGGGGYEKLF | 1.20% | 153 |
| | F (SEQ ID NO: 67) | | |
| clonotype10 | TRβ: CASSQAHGQNQPQH | 1.10% | 134 |
| | F (SEQ ID NO: 68) | | |

TABLE 7
TOP 10 CLONOTYPES IN SAMPLE - LP186-10-17

| Clonotype | | | |
|---|---|---|---|
| ID | CDR3s | Proportion | Frequency |
| clonotype1 | TRα: CAVTGTQGGKLIF | 3.00% | 171 |
| | (SEQ ID NO: 69) | | |
| | TRβ: CASSLGTGVSTEAF | | |
| | F (SEQ ID NO: 70) | | |
| clonotype2 | TRα: CILRDSNGANNLFF | 2.90% | 165 |
| | (SEQ ID NO: 71) | | |
| | TRβ: CASSPINRRNTEAF | | |
| | F (SEQ ID NO: 72) | | |
| clonotype3 | TRα: CIPWHLNDYKLSF | 2.90% | 163 |
| | (SEQ ID NO: 73) | | |
| | TRβ: CASSFQGSGNTIYF | | |
| | (SEQ ID NO: 74) | | |
| clonotype4 | TRα: CAASARTGANNLFF | 2.70% | 153 |
| | (SEQ ID NO: 75) | | |
| | TRβ: CASSDTSSYNSPLH | | |
| | F (SEQ ID NO: 76) | | |
| clonotype5 | TRα: CAVNKGYSTLTF | 2.50% | 144 |
| | (SEQ ID NO: 577) | | |
| | TRα: CVVRGLFSGGYNKL | | |
| | IF (SEQ ID NO: 78) | | |
| | TRβ: CASSSTGTGAAGEL | | |
| | FF (SEQ ID NO: 79) | | |
| clonotype6 | TRα: CAENSPNNAGNMLT | 2.10% | 118 |
| | F (SEQ ID NO: 80) | | |
| | TRβ: CASSQDAGNTEAFF | | |
| | (SEQ ID NO: 81) | | |
| clonotype7 | TRα: CALSENSGGGADGL | 1.90% | 108 |
| | TF (SEQ ID NO: 82) | | |
| | TRβ: CASSFTEYQETQYF | | |
| | (SEQ ID NO: 83) | | |
| clonotype8 | TRα: CVVNIGNYGQNFVF | 1.80% | 106 |
| | (SEQ ID NO: 84) | | |
| | TRβ: CASSASGTGGPRDT | | |
| | GELFF (SEQ ID NO: | | |
| | 85) | | |
| clonotype9 | TRβ: CASSYQTGASYGYT | 1.50% | 85 |
| | F (SEQ ID NO: 86) | | |
| clonotype10 | TRα: CVVNTDSWGKLQF | 1.40% | 80 |
| | (SEQ ID NO: 87) | | |
| | TRβ: CASSWDRGAGANVL | | |
| | TF (SEQ ID NO: 88) | | |

TABLE 8
TOP 10 CLONOTYPES IN SAMPLE - LP186-02-18

| Clonotype | | | |
|---|---|---|---|
| ID | CDR3s | Proportion | Frequency |
| clonotype1 | TRα: CARNTGNQFYF | 16.30% | 864 |
| | (SEQ ID NO: 89) | | |
| | TRβ: CASSYQTGASYGYT | | |
| | F (SEQ ID NO: 90) | | |
| clonotype2 | TRα: CARNTGNQFYF | 11.70% | 617 |
| | (SEQ ID NO: 91) | | |
| | TRβ: CASSPLTGTGVYGY | | |
| | TF (SEQ ID NO: 92) | | |
| clonotype3 | TRβ: CASSYQTGASYGYT | 8.50% | 451 |
| | F (SEQ ID NO: 93) | | |
| clonotype4 | TRβ: CASSPLTGTGVYGY | 5.40% | 285 |
| | TF (SEQ ID NO: 94) | | |
| clonotype5 | TRα: CVVNQAGTALIF | 3.40% | 182 |
| | (SEQ ID NO: 95) | | |
| | TRβ: CASSEQAFYEQYF | | |
| | (SEQ ID NO: 96) | | |
| clonotype6 | TRα: CAENSPNNAGNMLT | 3.10% | 165 |
| | F (SEQ ID NO: 97) | | |
| | TRβ: CASSQDAGNTEAFF | | |
| | (SEQ ID NO: 98) | | |
| clonotype7 | TRα: CARNTGNQFYF | 3.00% | 157 |
| | (SEQ ID NO: 99) | | |
| | TRβ: CASSYQTGAAYGYT | | |
| | F (SEQ ID NO: 100) | | |
| clonotype8 | TRα: CILTPTHNTDKLIF | 2.30% | 124 |
| | (SEQ ID NO: 101) | | |
| | TRβ: CASSLLRQTQYF | | |
| | (SEQ ID NO: 102) | | |
| clonotype9 | TRα: CAERLQTGANNLFF | 2.00% | 106 |
| | (SEQ ID NO: 103) | | |
| clonotype10 | TRβ: CASSYQTGAAYGYT | 1.50% | 77 |
| | F (SEQ ID NO: 104) | | |

With the above results, the inventors showed that they were able to analyze the α/β chains of the TCR from a single T cell. Reduced diversity was seen in clones in TIL population. On the other hand, a high diversity of clones in PBMCs from healthy subjects was observed. α/β chain information was found to be missing in some cells probably due to low level expression and/or work flow issues e.g fragmentation. The amplified cDNA library thus obtained can be used for transcriptional profiling of single cells to further characterize the T-cells.

### Example 2: Single Cell mRNA Sequencing/Profiling

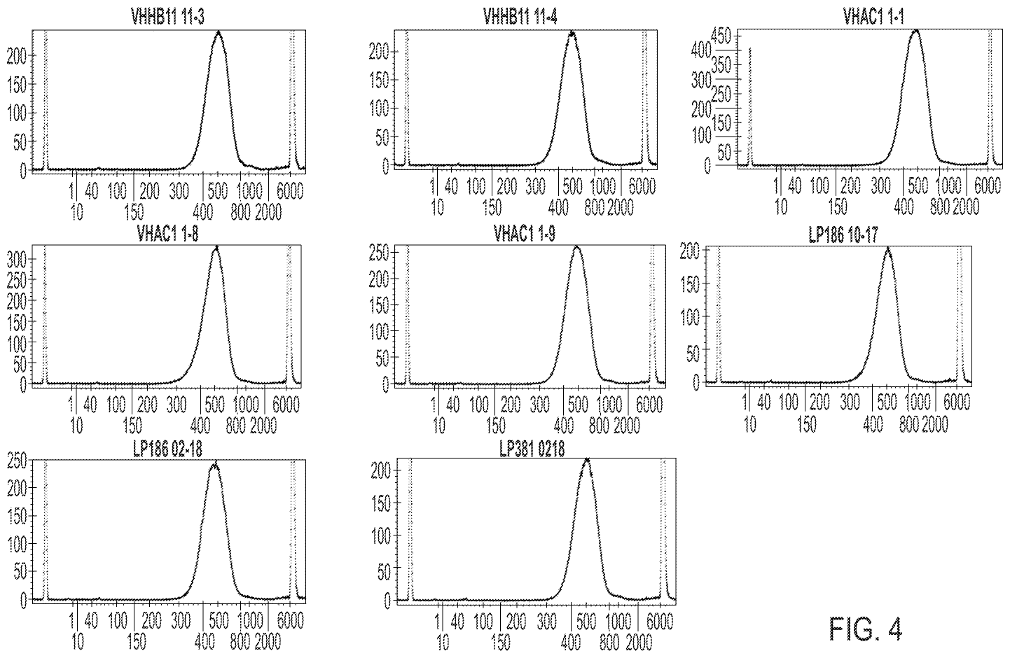

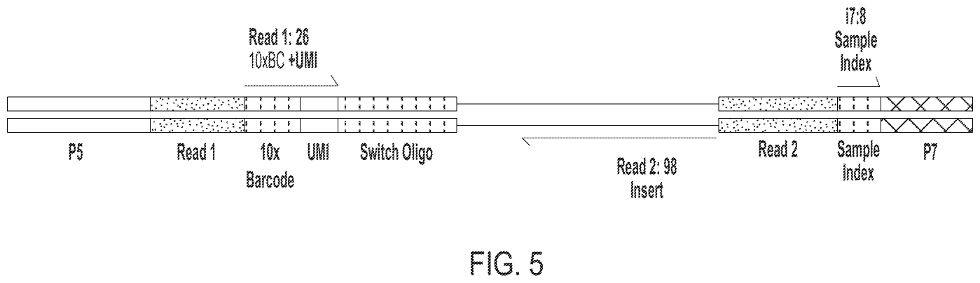

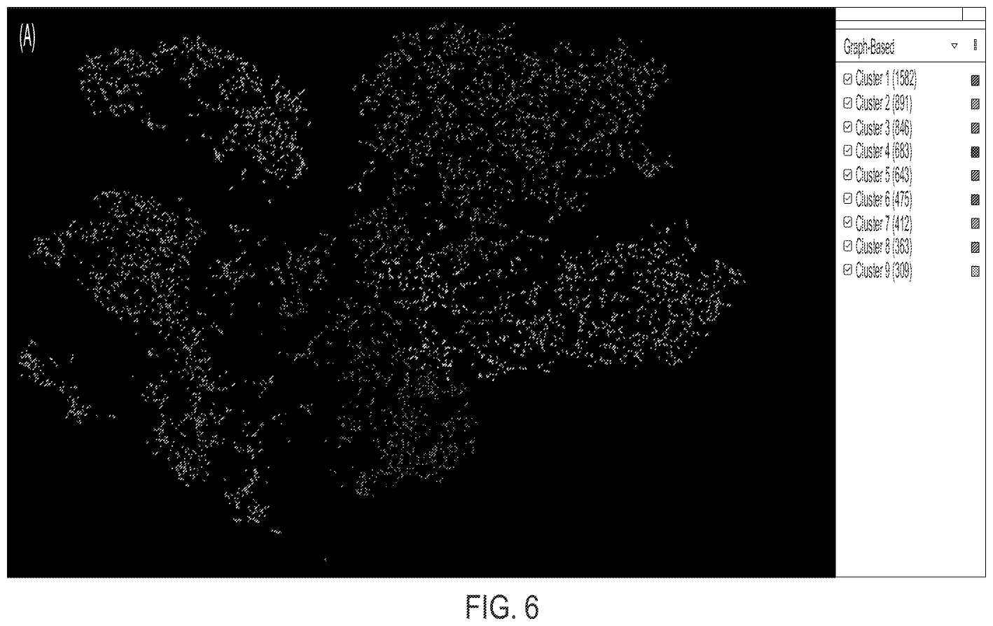

Referring to FIGS. 4 and 5, single cell mRNA sequencing/profiling was done, and GEX sequencing libraries for 8 samples are shown in FIG. 4, while FIG. 5 illustrates gene expression library structure. Table 9 below illustrates combined metrics for scRNA sequencing.

TABLE 9
Combined metrics for scRNA sequencing.

| Name of the | Estimated | Mean | Median | Total | Median |
|---|---|---|---|---|---|
| | No. of | Reads | Genes | Genes | UMI Counts |
| Sample | Cells | per Cell | per Cell | Detected | per Cell |
| LP186_10-17 | 5,038 | 64,578 | 2,545 | 20,138 | 8,958 |
| LP186_02-18 | 5342 | 66,368 | 2290 | 19,039 | 6,770 |
| LP381_02-18 | 5,454 | 75,859 | 2,207 | 19,160 | 6,258 |
| VHAC_1-1 | 6,784 | 61,291 | 2,743 | 19,163 | 8,511 |
| VHAC_1-8 | 7,413 | 57,993 | 2,859 | 20,033 | 11,077 |
| VHAC_1-9 | 12,286 | 31,849 | 1,874 | 19,157 | 5,179 |
| VHHB11_11-3 | 6,204 | 73,565 | 2,517 | 20,116 | 7,336 |
| VHHB11_1-4 | 7,289 | 59,311 | 2,374 | 20,088 | 6,600 |

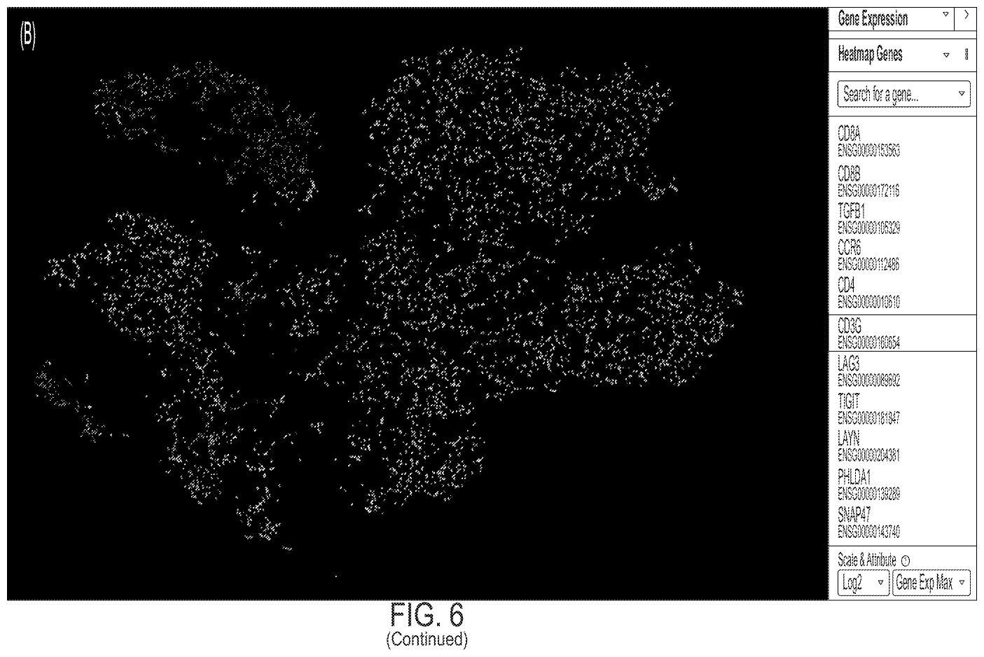

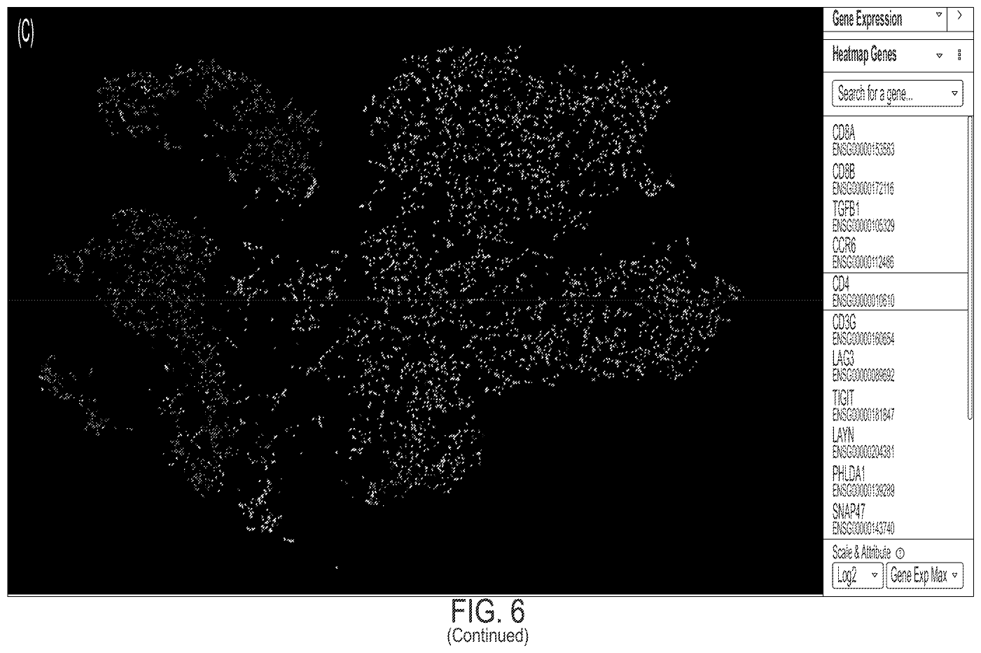

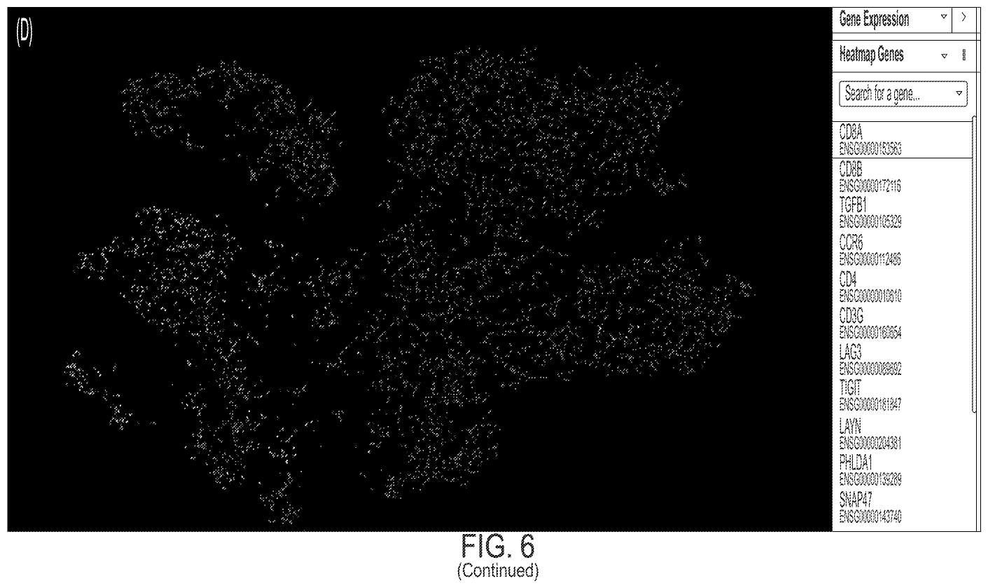

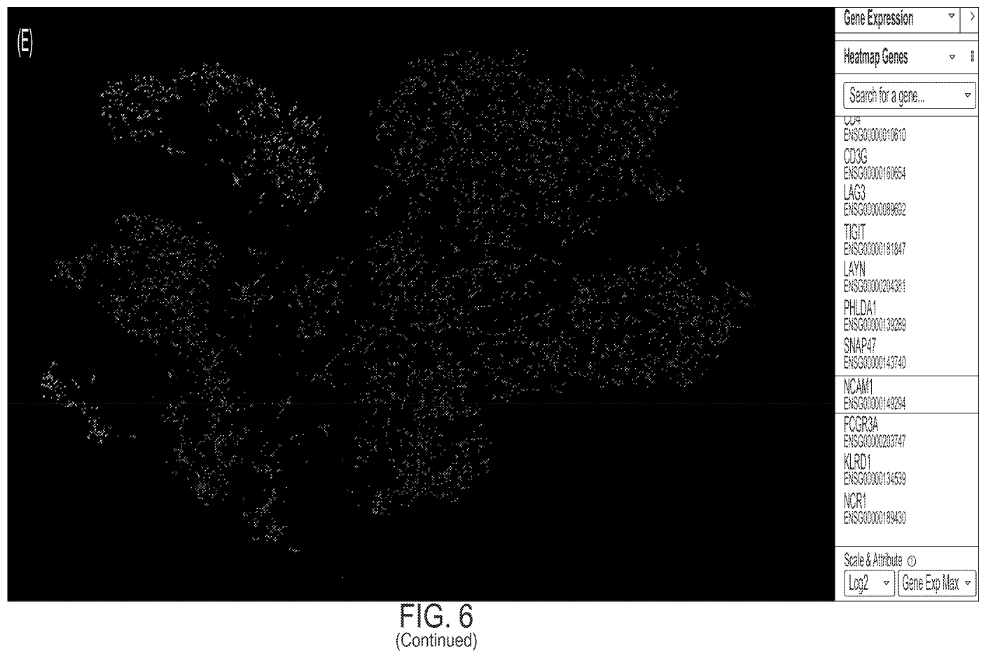

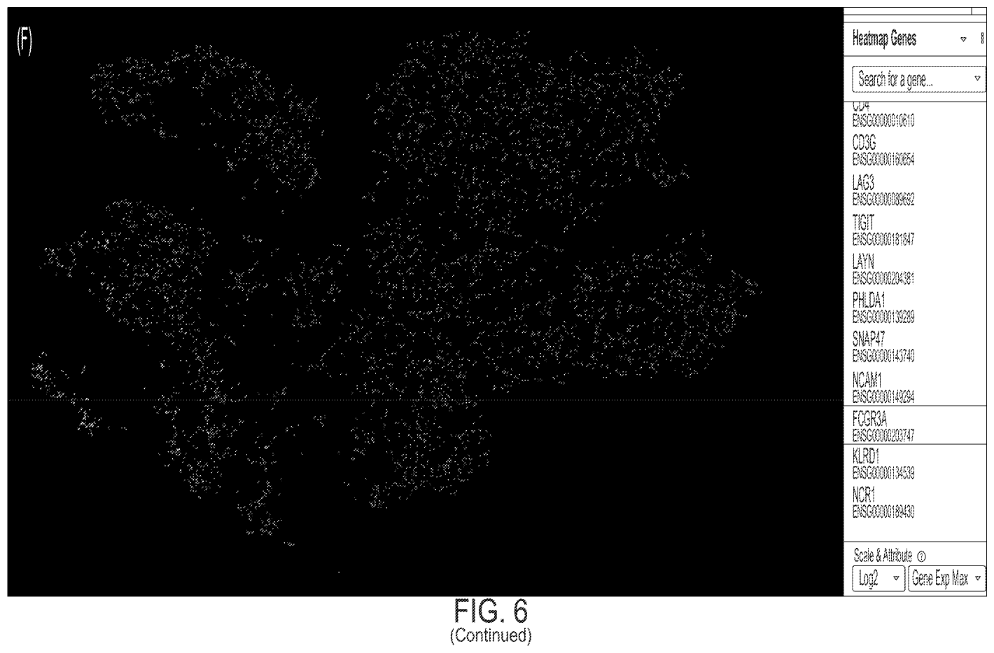

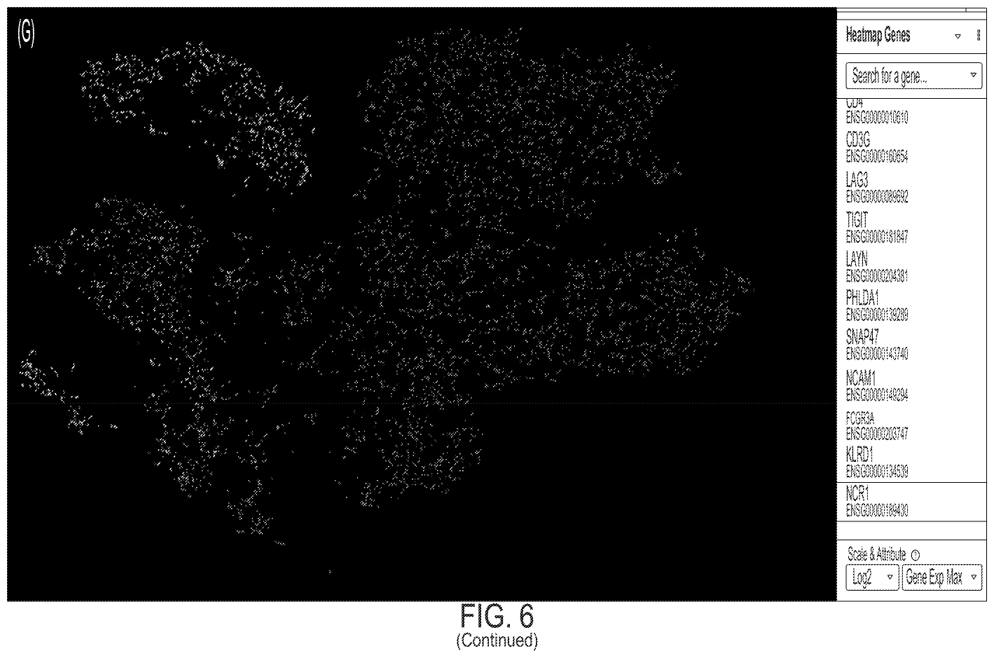

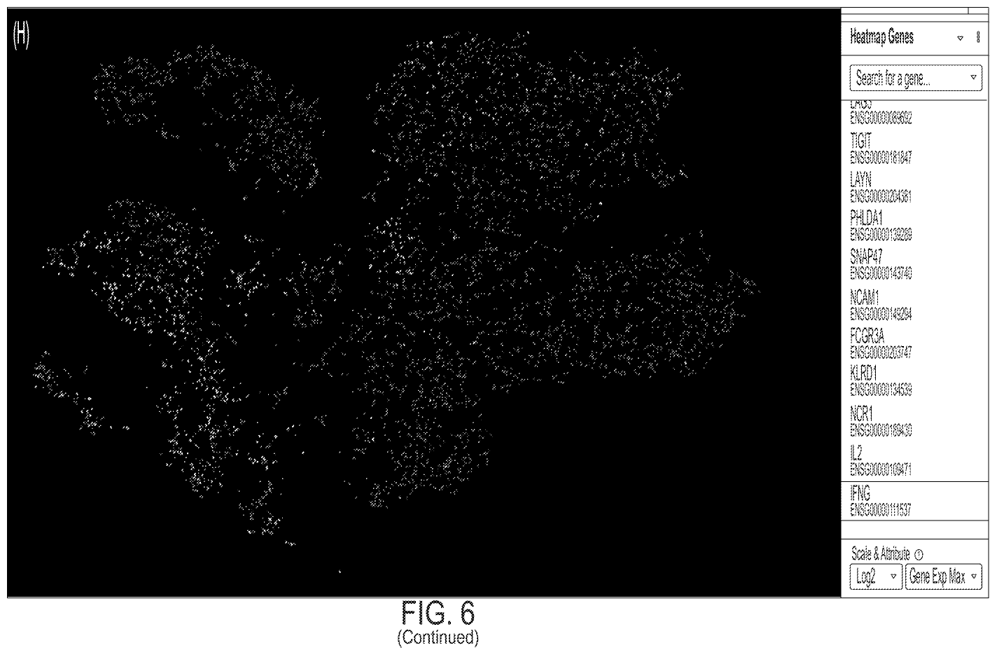

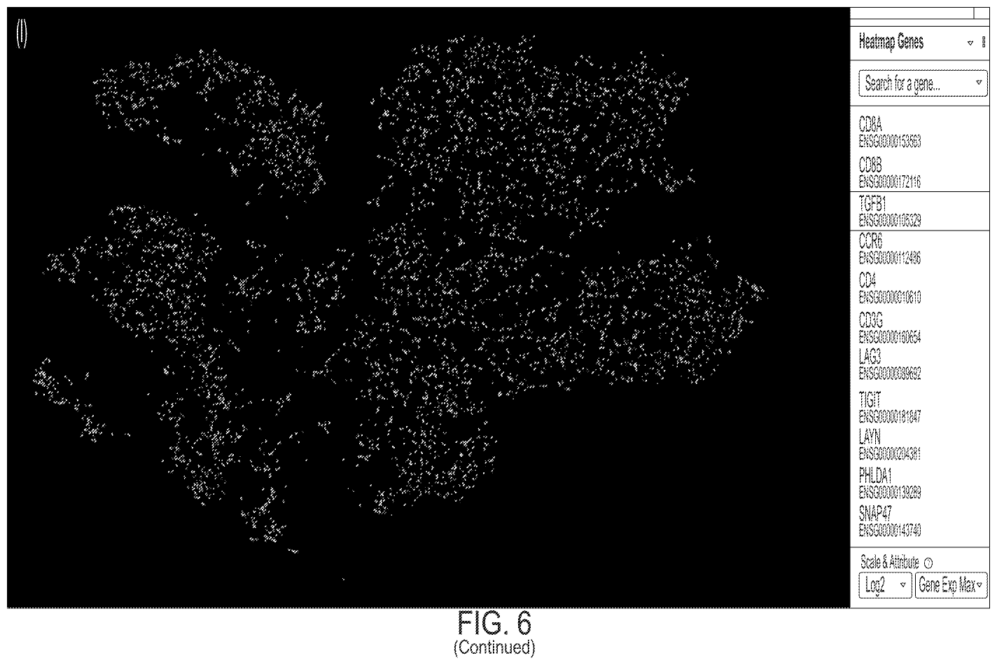

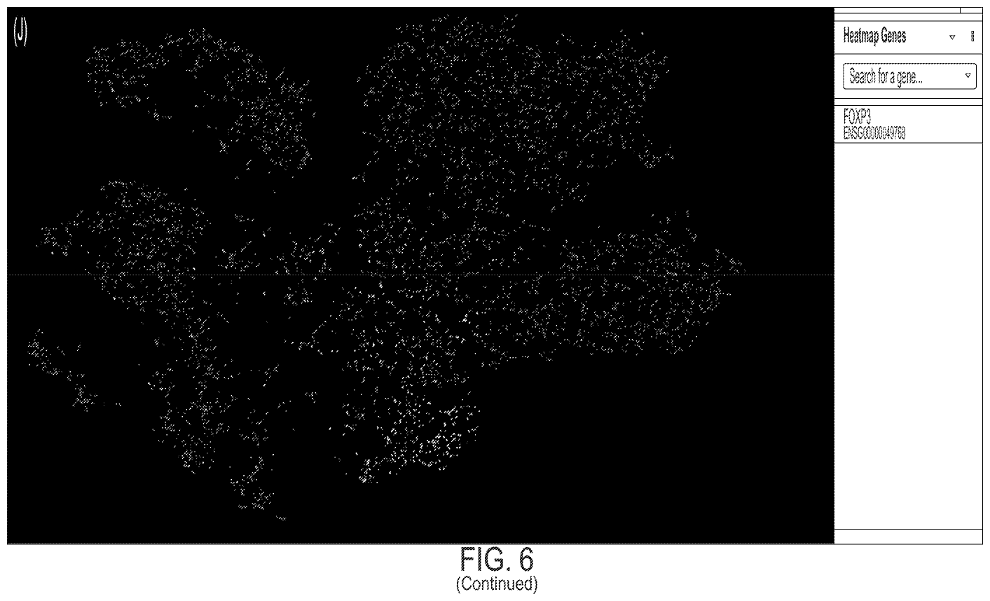

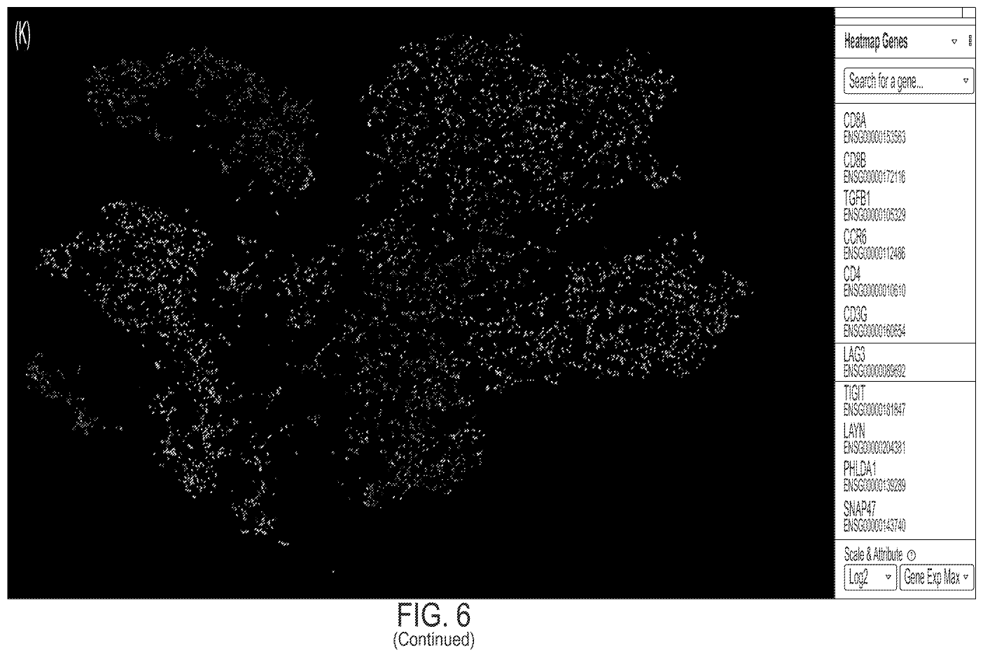

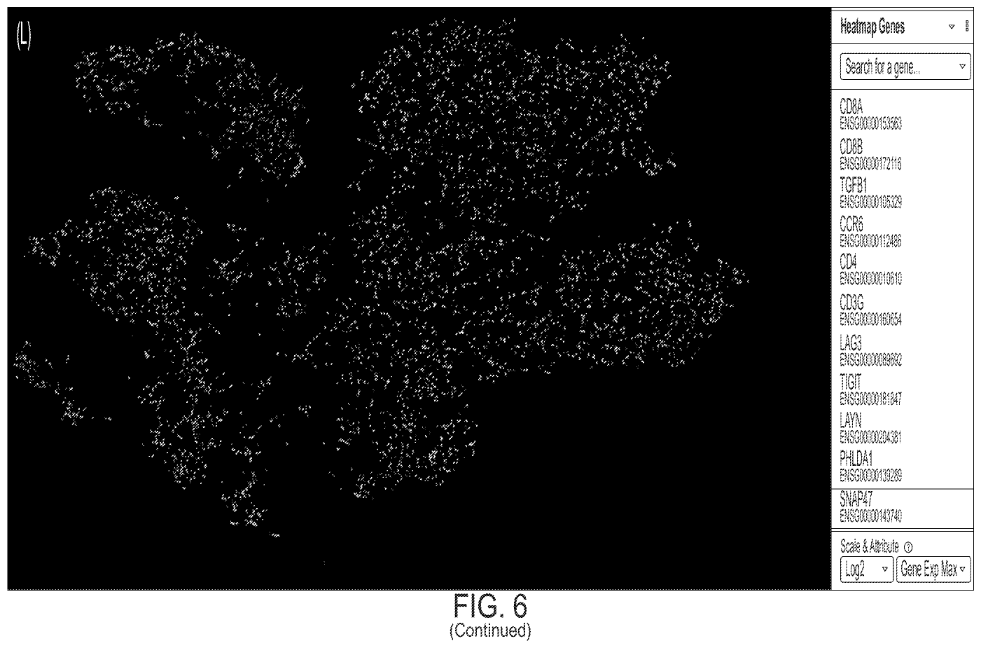

FIG. 6 illustrates single transcript analysis for 6204 cells 9 different clusters (FIG. 6A); 6204 cells CD3G (FIG. 6B), CD4 (FIG. 6C), CD8A (FIG. 6D), NCAM1 CD56 (FIG. 6E), FCGR3A CD16 (FIG. 6F), NCR1 NK-p46 (FIG. 6G), IFNγ (FIG. 6H), TGFβ1 (FIG. 6I), FOXP3 (FIG. 6J), LAG3 (FIG. 6K), and SNAP47 (FIG. 6L)

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of generating a treatment composition for a patient having a tumor, comprising:
preparing from a tumor tissue a plurality of single cells comprising single tumor cells and single immune competent cells;
using single cell nucleic analysis to determine from the plurality of single cells:
i. a T cell receptor profile for the immune competent cells;
ii. a first immune cell type profile; and
using the T cell receptor profile and the immune cell type profile to generate recombinant white blood cells,
wherein the recombinant white blood cells comprises T cell receptors targeting tumor associated antigens and neoepitopes and wherein the neoepitopes are determined by tumor-normal sequencing.

2. The method of claim 1, further comprising a step of generating a second immune cell type profile using peripheral white blood cells.

3. The method of claim 1, wherein the T cell receptor sequence information is obtained from a single cell RNA-seq, and comprises a RNA sequence encoding variable (V), joining (J), and optionally diversity (D) segments of the T cell receptor.

4. The method of claim 3, further comprising constructing a V(D)J library having a plurality of members, wherein each member comprises nucleic acid sequences encoding a barcode element, a unique molecular identifier (UMI), and a cDNA sequence reverse-transcribed from the RNA sequence.

5. A method of treating a patient having a tumor, comprising:
obtaining a set of T cell receptor sequence information from a tumor tissue and a normal tissue of the patient, wherein each of the T cell receptor sequence information corresponds to one or more T cell receptors expressed in a single T cell;
obtaining a set of single cell gene expression information from the tumor tissue and the normal tissue of the patient, wherein each of the single cell gene expression corresponds to gene expressions in a single while blood cell;
determining, from the set of T cell receptor sequence information, a molecular profile of T cells in the tumor tissue by comparing the T cell receptor sequence information of the tumor tissue with the T cell receptor sequence information of the normal tissue;
determining, from the set of single cell gene expression information, a molecular profile of white blood cells of the tumor tissue by comparing the single cell gene expression information of the tumor tissue with the and single cell gene expression information of the normal tissue;
determining an immunome of the tumor tissue based on the molecular profiles of T cells and the white blood cells of the tumor tissue; and
administering an immunotherapeutic composition comprising an immune competent cell that is genetically modified with a recombinant nucleic acid encoding a chimeric antigen receptor or a T cell receptor, wherein the recombinant nucleic acid comprises a nucleic acid segments encoding variable (V) and joining (J) segments selected based on the molecular profile of T cells.

6. The method of claim 5, wherein wherein the T cell receptor sequence information is obtained from a single cell RNA-seq, and comprises a RNA sequence encoding variable (V), joining (J), and optionally diversity (D) segments of the T cell receptor.

7. The method of claim 6, further comprising constructing a V(D)J library having a plurality of members, wherein each member comprises nucleic acid sequences encoding a barcode element, a unique molecular identifier (UMI), and a cDNA sequence reverse-transcribed from the RNA sequence.

8. The method of claim 5, wherein the molecular profile of T cells comprises at least one of number of cells expressing T cell receptor, a number of clonotype, and a frequency of the clonotype.

9. The method of claim 5, wherein the single cell gene expression information is obtained from single cell RNA-seq of a plurality of genes, each the gene encoding a protein in an immune response pathway.

10. The method of claim 9, further comprising constructing a gene expression library having a plurality of members, wherein each member comprises nucleic acid sequences encoding a barcode element, a unique molecular identifier (UMI), and a cDNA sequence reverse-transcribed from the RNA sequence of the plurality of genes.

11. The method of claim 10, wherein the molecular profile of white blood cells comprises a median number of genes expressed per cell, total number of detected genes, and median number of the unique molecular identifier.

12. The method of claim 11, further comprising clustering white blood cells in the tumor tissue into a plurality of clusters based on the molecular profile.

13. A method of profiling an immunome of a patient having a tumor, comprising:
obtaining T cell receptor sequence information from a tumor tissue and a normal tissue of the patient, wherein each of the T cell receptor sequence information corresponds to one or more T cell receptors expressed in a single T cell;
obtaining single cell gene expression information from the tumor tissue and the normal tissue of the patient, wherein each of the single cell gene expression corresponds to gene expressions in a single white blood cell;
determining, from the T cell receptor sequence information, a molecular profile of T cells in the tumor tissue by comparing the T cell receptor sequence information of the tumor tissue with the T cell receptor sequence information of the normal tissue;
determining, from the set of single cell gene expression information, a molecular profile of white blood cells of the tumor tissue by comparing the single cell gene expression information of the tumor tissue with the and single cell gene expression information of the normal tissue; and
determining an immunome of the tumor tissue based on the molecular profiles of T cells and the white blood cells of the tumor tissue.

14. The method of claim 13, wherein the molecular profile of T cells comprises at least one of number of cells expressing T cell receptor, a number of clonotype, and a frequency of the clonotype.

15. The method of claim 13, wherein the single cell gene expression information is obtained from single cell RNA-seq of a plurality of genes, each the gene encoding a protein in an immune response pathway.

16. The method of claim 15, further comprising constructing a gene expression library having a plurality of members, wherein each member comprises nucleic acid sequences encoding a barcode element, a unique molecular identifier (UMI), and a cDNA sequence reverse-transcribed from the RNA sequence of the plurality of genes.

17. The method of claim 16, wherein the molecular profile of white blood cells comprises a median number of genes expressed per cell, total number of detected genes, and median number of the unique molecular identifier.

18. The method of claim 17, further comprising clustering white blood cells in the tumor tissue into a plurality of clusters based on the molecular profile.

19. The method of claim 18, further comprising determining expressions of an immune cell marker gene.

20. The method of claim 19, wherein the immune cell marker gene comprises CD3G, CD4, CD8A, NCAM1 (CD56), FCGR3A (CD16), NCR1 (NK-p46), IFN-γ, TGF-β1, FOXP3, LAG3, and SNAP47.

21. The method of claim 13, further comprising:
creating an immunotherapeutic composition comprising an immune competent cell that is genetically modified with a recombinant nucleic acid encoding a chimeric antigen receptor or a T cell receptor; and
wherein the recombinant nucleic acid comprises a nucleic acid segments encoding variable (V) and joining (J) segments selected based on the molecular profile of T cells.

22. The method of claim 13, wherein the immune competent cell is a T cell, an NK cell, a genetically engineered NK cell, or an NKT cell.

23. The method of claim 13, further comprising administering a plurality of immune competent cells to the patient, wherein types of the plurality of immune competent cells are selected based on the molecular profile of the white blood cells.

24. The method of claim 23, wherein at least one of the immune competent cells is the patient's autologous cell.
